(19) United States
(12) Patent Application Publication
(10) Pub. No.: US 2011/0060048 A1
(43) Pub. Date: Mar. 10, 2011

(54) Removing alcohol in vivo through esterification

(76) Inventor: Ashley Regina Lo, San Diego, CA (US)

(21) Appl. No.: 12/554,899

(22) Filed: Sep. 5, 2009

(51) Int. Cl. A61K 31/19; A61K 31/194

(52) U.S. Cl. 514/574; 514/557

(57) ABSTRACT

The present invention is pertaining to a method to chemically remove ethanol in the stomach before a significant amount of ethanol enters the blood stream. The key chemical reaction responsible for ethanol removal is esterification through a reaction between ethanol and acetic acid. Ethanol reacts with acetic acid to produce water and ethyl acetate, which is a harmless ester found in many food products. In theory, if an intoxicated individual consumes a molar quantity of vinegar similar to the amount of ethanol in the alcoholic beverage, much of the ethanol will be converted into harmless products. Low cost, practical methods for alcohol drinkers to orally take a sufficient amount of acetic components are invented. These methods include drinks containing a mixture of acetic acid and potassium hydroxide with an appropriate level of acidity.

### BACKGROUND

Ethanol (C₂H₅OH) is a depressant that is present in all alcoholic beverages in varying percentages. On an empty stomach, 20% of the ethanol consumed almost immediately diffuses into the bloodstream through the walls of the stomach. Within 30 minutes, all of the ethanol will have entered the blood, and clear symptoms can be observed if the BAC (blood alcohol content) of the drinker is over 0.08. Years of chronic drinking leads to lasting damage on vital organs.

Effects of alcohol on various parts of the body are summarized below:
1. The liver: The majority of alcohol consumed is transported to the liver to be converted into harmless products. As a result, 15% of all alcohol-related deaths are attributable to liver failure. Liver damage begins with the hepatotoxic effect (aka fatty liver), and inflicts moderate, reversible damages on the liver. If drinking continues, scarring and inflammation will deteriorate, and eventually culminate in liver cirrhosis, which is irreversible and usually fatal.
2. The brain: Short term effects include drowsiness, loss of coordination, dizziness, and mood swings. Long-term effects include memory loss, strokes, and concussions.
3. Reproductive system: In males, alcohol abuse results in prostate inflammation and erectile disorder. In women, it results in irregular menstrual cycles, hormonal imbalance, infertility, and—if pregnant—physical deformities and mental retardation in the fetus.
   Alcoholism has far-reaching ramifications on both individuals and society. Although it is impractical to forbid people from drinking altogether, there is a need to limit its effects.

### Prior Arts:

In January 2008, a patent was filed under the name “Method for the Accelerated in Vivo Removal of Ethanol” (U.S. Pat. No. 7,323,452). The patent stated that the liver naturally converts ethanol into acetaldehyde using an enzyme called alcohol dehydrogenase, but that the equilibrium constant of 1 indicated inefficiency. It suggested that a pill, capsule, or nasal spray containing chemicals accelerators in the direction of acetaldehyde (such as zinc ions) could be added in order to increase the proportion of ethanol that was being eliminated. However, this proposal is based on the assumption that ethanol has already entered the bloodstream, and must be eliminated from there. By the time ethanol has entered the bloodstream, it is extremely difficult to eliminate it in large quantities. Ethanol is a very small molecule (molar mass 46 g/mol) compared to the organic macromolecules, and even molecules such as glucose and amino acids, that normally circulate throughout the bloodstream. Its solubility in water additionally allows it to circulate rapidly throughout the body. Clearly, the removal process must take before ethanol enters the bloodstream.

In 1984, a patent was filed under the title of “Alcohol removal from blood with alcohol oxidase (U.S. Pat. No. 4,450,153). Alcohol oxidase is an enzyme that converts ethanol into acetaldehyde in the following method: primary alcohol+O₂⇄an aldehyde+H₂O₂. After the aldehyde is produced, it naturally decays into water and carbon dioxide, which are eliminated through urination and respiration. However, the alcohol oxidase does not naturally exist in the human body in sufficient amounts, and is very costly. A solution containing approximately 17,200 EU of alcohol oxidase is needed to neutralize the ethanol in a can of beer within 15 minutes. This amount of enzyme will cost nearly $370. A method that requires nearly four hundred dollars to reverse the effects of a single serving of a mild alcoholic beverage cannot be applied commercially, or developed into an easily-accessible sobriety drug.

Another method was published in a 1975 volume of the American Journal of Clinical Nutrition, for removing ethanol from the body through ingesting sugars. Eight male volunteers 25-50 years old, of approximately the same height and weight, were each given a few servings of alcoholic beverage, followed by 30 grams of fructose or sucrose. Measurements were taken each hour, and the percent of ethanol decrease flattened out at 29% on average. This experiment is dubious, because a). It only eliminated 29% of the ethanol from the bodies of the drinkers, and thus does not appear to be a very efficient antidote, b). Fructose is very expensive, and c). It promotes a high-sugar diet.

As will be discussed below, the use of the acetate ion to neutralize ethanol is a much more efficient method.

### SUMMARY OF INVENTION

The key principle of this invention for ethanol removal is ethanol esterification with acetic acid, as described in the following reaction:

C₂H₅OH+CH₃COOH→←CH₃CH₂OCOCH₃K_{eq}=45 M⁻¹

In an in-vitro test, a known volume (17.15 mL) and concentration (0.4 M, 0.6 M, 0.8 M, 1.0 M) of ethanol reacted with the same volume and concentration of acetic acid. Titrations with potassium permanganate (KMnO₄) were later performed after the contents of each beaker received 5 minutes each of vigorous stirring and 25 minutes of inactivity to compare the final amount of ethanol left with the original amount. In agreement with the calculations done using the known equilibrium constant of K=45 M⁻¹, a 79-86% decrease in the amount of ethanol was demonstrated when the system reaches equilibrium. Average results calculated after 5 trials were within a 4% margin of error, as shown in Table 1.

TABLE 1
In vitro experiment of esterification with different values of initial
ethanol and acetic acid concentrations. The last column indicates
the amount of decrease in the ethanol concentration after the
system reaches equilibrium.

| Initial | | | |
|---|---|---|---|
| C₂H₅OH = | | | |
| Initial | Equilibrium Concentrations | | % Ethanol |
| CH₃COOH | C₂H₅OH═CH₃COOH | CH₃CH₂OCOCH₃ | Decrease |
| 0.4 M | 0.084 M | 0.316 M | 79.0% |
| 0.5 M | 0.095 M | 0.405 M | 81.0% |
| 0.6 M | 0.105 M | 0.495 M | 82.5% |
| 0.7 M | 0.114 M | 0.586 M | 83.7% |
| 0.8 M | 0.123 M | 0.677 M | 84.6% |
| 0.9 M | 0.131 M | 0.769 M | 85.4% |
| 1.0 M | 0.138 M | 0.862 M | 86.2% |

The amount of alcohol intake before reaching the DUI level could vary widely among individuals and depends on many factors such as weight, age, gender, and race. It is generally believed that, for most people, four cans of beer or an equivalent amount of ethanol taken over a time period of 1-2 hours can influence people's health, behaviors, and judgment. Four cans of beer contain approximately 1 mole of ethanol. According to the above in vitro experiment, it will take about an equal amount (i.e. 1 mole) of acetic acid to substantially reduce the amount of ethanol through esterification. A 100 ml of off-the-counter vinegar (5% acidity), on the other hand, contains only 0.086 mole of acetic acid. It is unconceivable that anyone is able to take 1160 ml of off-the-shelf vinegar in order to effectively remove the ethanol. Both the sheer volume and the acidity of the vinegar make the approach impractical.

An important part of the invention is about practical methods to orally take the required amount of acetic acid to react with ethanol. A practical approach has to meet two requirements: (a) substantially reduce the intake volume of acetic acid containing liquid, and (b) introducing the required amount (around 1.0 mole) of acetic acid in a harmless and least intrusive manner, meaning to create no acid burn or discomfort. In accordance with the invention, these practical approaches are developed based on one key concept: reducing the acidity of concentrated acetic acid with a proper amount of strong base to yield a final product having a proper level of acidity, i.e. for a range of PH value between 3-4. For references, the PH value is 2.9-3.3 for apple juice, 3-4 for orange juice, and 4.2 for tomato juice. Possible choices of strong base include NaOH and KOH. Added to concentrated acetic acid, the solution contains CH₃COONa or CH₃COOK and CH₃COOH with lower acidity. The mixed solution contains sodium and/or potassium, and the amount should be kept well below the FDA recommended daily allowance. Since it is less desirable to create high sodium-containing drinks, KOH becomes a more attractive choice of base to partially neutralize the acetic acid.

The detailed analysis, to be discussed in the following section, suggests the following practical recipe:

Adding 0.02 mole of KOH (20% FDA recommended daily dosage for potassium) to 1 mole of 50% concentrated acetic acid, one can take 1 mole of acetic acid in a 75 mil drink at a PH-value of around 3.3.

Numerous recipes following the similar principle can be created to produce essentially the same effect of turning ethanol into ester in stomach. Additional flavors and components can also be introduced to the drink without altering its function while improving the taste. Furthermore, the same ingredients can also be incorporated into solid food such as cakes, snack bars, cookies, and other bakeries in manners that the heating process of making such goods will not degrade the performance. It is also conceivable that the mixed KOH/CH₃COOH solution may be turned into powder or gel with extra additives so that it can be packaged into pills that are easier to carry.

The invention produces a solution for removing the harmful effects of alcohol and is inexpensive, safe and effective. Compared to approaches that use enzymes, acetic acid and bases such as KOH and NaOH are easy to obtain in large quantities at very low cost. In addition, all ingredients (CH₃COOK and CH₃COOH) are safe to use and their daily dosages (e.g. K and Na) are well established. Therefore, the product can be off the counter without prescription. It does not require FDA approval either.

Although we have used acetic acid as an example to turn the harmful ethanol into a product that is more benign, one can choose other organic acid such as citric acid to achieve the similar effect. Each citric acid molecule contains three functional sites to react with three ethanol molecules to generate a product of triethylcitrate. Since citric acid is a weak acid too, the above mentioned technique of partial neutralization with strong base is applicable too. It should be understood that all approaches based on the similar principle discussed in this disclosure are covered by this invention.

### DETAILED DESCRIPTION OF INVENTION

The first part of the invention is the process of turning ethanol into ester from an oral intake of acetic acid. The reaction of esterification occurs inside stomach. The second part of the invention is the methodology of making oral intake products that contain a sufficient amount (e.g. 1 mole) of acetic acid in a relatively small volume (e.g. 75 mil) of proper acidity (e.g. PH: 3-4).

In the following we describe the detailed procedure of in vitro experiment to verify the validity of reducing the amount of ethanol by esterification:
1. Label four 100-mL beakers, 0.4 M, 0.6 M, 0.8 M, and 1.0 M.
2. Put 17.15 mL of distilled water into each.
3. Add 0.4 mL of concentrated ethanol to the first beaker, 0.6 mL to the second, 0.8 mL to the third, and 1.0 mL to the fourth. Swivel each beaker gently for 20-30 seconds to ensure mixing. See calculations below for explanation.
4. Add another 17.15 mL of water to each beaker
5. Add 0.4 mL of glacial acetic acid to the first beaker, 0.6 mL to the second, 0.8 mL to the third, and 1.0 mL to the fourth. See calculations below for explanation.
6. Stir each sample vigorously for five minutes in succession. Each beaker will have had a total of twenty minutes for the reaction to proceed.
7. Put on latex gloves before handling potassium permanganate.
8. Set up two burettes, and fill each with approximately 100 mL of 0.2 M potassium permanganate. This will enable two titrations to be simultaneously done.
9. Record the initial volume of potassium permanganate present in both burettes.
10. Place 1.0 M and 0.8 M beakers on hot plate, and turn to power level 3. Heating quickens the titration process.
11. Slowly drip KMnO₄ into beakers while stirring. Make sure that each beaker is consistently titrated with the same burette to ensure that the exact volumes of KMnO₄ that each beaker received can be easily calculated.
12. Potassium permanganate is purple in color. When reacted with ethanol, a brown color (manganese oxide, MnO₂) appears. The reaction is as follows:

3C₂H₅OH+4KMnO₄→3CH₃COOH+4MnO₂+4KOH+H₂O

When potassium permanganate ceases to be converted into manganese oxide, and the purple color stays, the titration is finished. There is no ethanol left in the beaker to react with potassium permanganate.
13. Remove both beakers from hot plate. Measure the volume of potassium permanganate remaining in each burette. Subtract from the original volumes to find the volume of KMnO₄ consumed in each reaction.
14. Repeat steps 9-13 for the 0.6 M and 0.4 M beakers.
15. Use stoichiometry to calculate the molar amount of 0.2 M potassium permanganate used in each titration.
16. Ethanol and potassium permanganate react in a 3:4 molar ratio, according to the equation 3C₂H₅OH+4KMnO₄→3CH₃COOH+4MnO₂+4KOH+H₂O.

Again, use a chain calculation to calculate the molar amount of ethanol remaining in each beaker, then divide by 0.01715 mL H₂O to convert to molarity. Compare to the molarity of ethanol originally in each beaker. Calculate % error.
17. Results (see Table 2)

TABLE 2
Summary of the results of 5 repeats of in vitro experiment.
The last column indicates the amount (in percentage) of reduction
in ethanol with the addition to acetic acid.

| Initial | C₂H₅OH % Decrease at Equilibrium | | | | | |
|---|---|---|---|---|---|---|
| C₂H₅OH | Trial 1 | Trial 2 | Trial 3 | Trial 4 | Trial 5 | Average |
| 0.4 M | — | 78.1% | 81.5% | 81.2% | 80.0% | 80.2% |
| 0.6 M | — | 82.7% | 83.8% | 83.7% | 82.5% | 83.2% |
| 0.8 M | 89.1% | 86.2% | 84.7% | 84.6% | 85.5% | 86.0% |
| 1.0 M | 91.3% | 88.8% | 86.5% | 91.1% | 90.4% | 89.6% |

Table 2 summarizes the results of 5 repeats of experiment. The results indicate that over a wide range of ethanol concentration relevant to our application, adding an equal molar number of acetic acid to the ethanol solution can reduce the ethanol amount by as much as 80-90%.

The second part of the invention is regarding to the methodology of creating products that contain a sufficient amount (e.g. 1 mole) of acetic acid in a relatively small volume (e.g. 75 mil) with proper acidity (e.g. PH: 3-4). The design principle and analysis are discussed in the following:

We use KOH as an example to reduce the acidity of a large amount of CH₃COOH so that one can comfortably drink enough amount of acetic acid by mouth to remove alcohol in the stomach.

The chemical reaction for dissociation of CH₃COOH:

$${CH}_{3} COOH \to {CH}_{3} {COO}^{-} + {H}^{+} \\ {K}_{α} = \frac{[ {CH}_{3} {COO}^{-} ] [ {H}^{+} ]}{[ {CH}_{3} COOH ]} ; {K}_{a} = 1 \times {10}^{- 5} M \qquad ( 1 )$$

If we do not add any KOH, then the PH value of CH₃COOH becomes

$${PH}_{{CH}_{3} COOH} ≅ - {log}_{10} \sqrt{{K}_{α} X} = 2.5 - \frac{1}{2} {log}_{10} X \qquad ( 2 )$$

Where X is the molar concentration of CH₃COOH. For 50% acetic acid, X=13.5 M. From Eq. (2), the acetic solution has a PH value of 2.0. Note that this is 10 times stronger acetic acid than the off-the-counter white vinegar and the solution is corrosive and could cause acid burn, not suitable for oral intake.

For 0.1% highly diluted vinegar, X≈0.03 M, the PH value, according to Eq. (2), becomes 3.3. This is close to the PH value of apple juice and orange juice, a PH value we are used to in food and beverage. Our approach is to create an acetic solution containing a large enough amount of acetic acid while keeping the PH value at the level of fruit juice.

Next we calculate how the PH value of the acetic solution changes with the addition of a small amount of KOH. Before reaction, we assume the initial molar concentration of CH₃COOH and KOH is X and Y, respectively. We also assume that we have a large amount of CH₃COOH so the final PH value of the solution is slightly acidic. This assumption works for our purpose because we try to maximize the amount of CH₃COOH but control the amount of KOH so the potassium intake can be kept well within the FDA recommended daily dosage (i.e. 4 g or 0.1 mole of potassium).

After equilibrium is reached, the final concentration for each component becomes:

$$\begin{matrix}[ {CH}_{3} COOH ] = X - u , [ {CH}_{3} {COO}^{-} ] = u , [ {H}^{+} ] = u - Y > 0 & \\ [ {H}^{+} ] = \frac{{K}_{α} [ {CH}_{3} COOH ]}{[ {CH}_{3} {COO}^{-} ]} ⇒ u - Y = \frac{{K}_{α} ( X - u )}{u} & ( 3 )\end{matrix}$$

Since acetic acid is a weak acid with a very low value of Kₐ<<1, we can find an approximate solution for u:

$$u ≅ \frac{Y}{2} + \frac{\sqrt{{Y}^{2} + 4 {K}_{α} X}}{2} \qquad ( 4 )$$

Then the PH value can be represented as

$$PH = - {log}_{10} [ \frac{\sqrt{{Y}^{2} + 4 {K}_{α} X}}{2} - \frac{Y}{2} ] ≅ - {log}_{10} [ \frac{{K}_{α} X}{Y} ] = 5 - {log}_{10} [ \frac{X}{Y} ] \qquad ( 5 )$$

Table 3 shows the PH value of the solution for different concentration ratios between CH₃COOH and KOH before mixture.

TABLE 3
Dependence of PH value of the solution on the ratio of
CH₃COOH and KOH before reaction.

| X/Y: initial ratio of [CH₃COOH] and [KOH] | PH value at equilibrium |
|---|---|
| 10 | 4.0 |
| 50 | 3.3 |
| 100 | 3.0 |

This result is highly significant. It shows that we can mix KOH with 50 times as much as CH₃COOH while maintaining an acidity level comfortable for drinking (i.e. equivalent to 0.1% highly diluted acetic acid).

The daily recommended potassium (K) intake is 4 g, equivalent to 0.1 moles. That means the above approach allows us to take as many as 5 moles of acetic acid without feeling acid burn or discomfort! 5 moles of acetic acid is much more than we need for ethanol esterification since 4 cans of beer contain only a total amount of 1.0 mole of ethanol.

While the invention has been described in connection with various embodiments, it is not intended to limit the scope of the invention to the particular form set forth. It is intended to cover such alternatives, modifications, and equivalents as may be included within the spirit and scope of the invention as defined by the appended claims

We claim:

1. A process for in vivo esterification of ethanol, comprising the steps of
(a) preparing a composition containing acetic acid and a base wherein the base partially neutralizes the acetic acid to a PH value of greater than 2.5; and
(b) administering the composition in vivo to reduce the amount of ethanol by turning a certain amount of ethanol into ester; and
(c) the reaction of esterification occurs mostly within stomach in an acetic environment and in the presence of stomach juice.

2. The process of claim 1, wherein the base contains potassium hydroxide.

3. The process of claim 2, wherein the amount of potassium is less than 8 grams or 200% of FDA recommended daily dosage.

4. The process of claim 1, wherein the base contains sodium hydroxide.

5. The process of claim 4, wherein the amount of sodium is less than 4.6 grams or 200% of FDA recommended daily dosage.

6. The process of claim 1, wherein the base contains calcium hydroxide.

7. The process of claim 1, wherein the composition is in liquid form.

8. The process of claim 1, wherein the composition contains food color, or food flavor, or other ingredients to make the composition more palatable to take orally.

9. The process of claim 1, wherein the composition is solidified with additional process and additives.

10. The process of claim 5, wherein the liquid is mixed with other ingredients to become a form of solid food or pill that can be taken in by mouth.

11. A composition to be taken orally to reduce the amount of ethanol inside body
(a) the composition contains at least 0.1 mole of acetic acid;
(b) the composition contains a base solution to increase the overall PH value above 2.5;
(c) the acetic acid in the composition can react with ethanol to form ester in vivo;
(d) the base in the composition contains ions of an alkali or alkaline earth metal.

12. The composition of claim 11, wherein the alkali metal is potassium.

13. The composition of claim 11, wherein the alkali metal is sodium.

14. The composition of claim 11, wherein the alkaline earth metal is calcium.

15. The composition of claim 11, wherein the alkaline earth metal is magnesium.

16. The composition of claim 11, wherein the composition is in liquid form.

17. The composition of claim 11, wherein the composition contains food color, or food flavor, or other ingredients to make the composition more palatable to take orally.

18. The composition of claim 11, wherein the composition is solidified with additional process and additives.

19. The composition of claim 11, wherein the liquid is mixed with other ingredients to become a form of solid food or pill that can be taken orally.

20. A composition to be taken orally to reduce the amount of ethanol inside body
(a) the composition contains at least 0.1 mole of organic acid;
(b) the composition contains a base solution to increase the overall PH value above 2.5;
(c) the composition can react with ethanol to form ester in vivo;
(d) the base in the composition contains ions of an alkali or alkaline earth metal.

21. The composition of claim 20, wherein the organic acid is citric acid.

22. The composition of claim 20, wherein the alkali metal is potassium.

23. The composition of claim 20, wherein the alkali metal is sodium.

24. The composition of claim 20, wherein the alkaline earth metal is calcium.

25. The composition of claim 20, wherein the alkaline earth metal is magnesium.

26. The composition of claim 20, wherein the composition is in liquid form.

27. The composition of claim 20, wherein the composition contains food color, or food flavor, or other ingredients to make the composition more palatable to take by mouth.

28. The composition of claim 20, wherein the composition is solidified with additional process and additives.

29. The composition of claim 20, wherein the liquid is mixed with other ingredients to become a form of solid food or pill that can be taken orally.
